SEQUENCE LISTING (3 sequences)

SEQ ID NO: 1 - DNA, 21 bp, Artificial Sequence
  Synthetic Polynucleotide
aacctggacatacgcatccga

SEQ ID NO: 2 - DNA, 20 bp, Artificial Sequence
  Synthetic Polynucleotide
cggatgcgtatgtccaggtt

SEQ ID NO: 3 - DNA, 21 bp, Artificial Sequence
  Synthetic Polynucleotide
cgactcactatagggagatgt

(19) United States
(12) Patent Application Publication
(10) Pub. No.: US 2017/0204403 A1
(43) Pub. Date: Jul. 20, 2017

(54) PROCESS FOR PRODUCING BACTERIAL MUTANTS

(71) Applicant: DISCUVA LIMITED, Cambridge (GB)

(72) Inventors: David Hugh Williams, Melbourn (GB); John Richard Wain, Melbourn (GB); Stuart Robert Woods, Longstanton (GB)

(73) Assignee: DISCUVA LIMITED, Cambridge (GB)

(21) Appl. No.: 15/415,357

(22) Filed: Jan. 25, 2017

Related U.S. Application Data
(63) Continuation of application No. PCT/GB2015/052080, filed on Jul. 17, 2015.

(30) Foreign Application Priority Data
Jul. 25, 2014 (GB) 1413202.1

(51) Int. Cl. C12N 15/10; C12Q 1/68

(52) U.S. Cl. CPC C12N 15/1058; C12N 15/1082; C12Q 1/689; C12Q 2600/158

(57) ABSTRACT

Disclosed is a process for producing a mutant bacterium which exhibits improved survival and/or growth under a selected growth condition, the process comprising the steps of: (a) generating a pool of mutant bacteria by transposon mutagenesis with an activating transposon (TnA), wherein the TnA comprises an outward-facing promoter (TnAP) capable of increasing transcription of a gene at or near its insertion site in the DNA of said bacterium; (b) growing bacteria from the mutant pool under the selected growth condition and under one or more reference conditions to produce two or more test cultures; and (c) sequencing mRNA transcripts produced by TnAP in each of said test cultures to produce an mRNA transcript profile for each of the test cultures; and (d) comparing the mRNA transcript profiles of the test cultures to identify a first class of genes which are disadvantageous for growth and/or survival under the selected growth condition and a second class of genes which are advantageous for growth and/or survival under the selected growth condition.

### RELATED APPLICATIONS

This application is a continuation of, and claims the benefit of priority to, international application PCT/GB2015/052080, filed Jul. 17, 2015, which was published under PCT Article 21(2) in English, and which claims priority to United Kingdom application 1413202.1, filed Jul. 25, 2014, the entire contents of each of which are incorporated by reference herein.

### FIELD OF THE INVENTION

The present invention relates to processes for engineering bacterial cells for use in biotechnological applications, including the production of proteins, secondary metabolites and biofuels, biocatalysis, bioremediation, biotransformation, biodegradation, biological control, drug development, drug screening, vaccines, probiotics, biosensors and drug delivery vehicles.

### BACKGROUND TO THE INVENTION

Bacteria find application in many aspects of biotechnology, including use as hosts for the production of heterologous proteins and peptides (including enzymes and therapeutic antibodies), as the source of secondary metabolites or their derivatives, as agents for biological control, crop production, biodegradation, biofuel production, biocatalysis and bioremediation and as probiotics, vaccine components and drug delivery systems.

Naturally occurring bacterial strains are not optimized for biotechnological use. One of the aims of the emerging field of synthetic biology is the engineering of new organisms which are more tractable and/or more efficient as biotechnological tools.

One approach may be termed the “ground-up” approach (see e.g. WO2008/024129). This involves the synthesis of a minimalized, artificial bacterial DNA genome containing only those genes essential for growth in any condition. This is then used to create a bare “chassis” to which desired biosynthetic pathways, signalling pathways or catabolic functions can be added as required and/or growth in commercially useful conditions can be optimised. This approach is currently impractical, since gene products and regulatory elements synergize and cross-talk in the context of the whole cell in ways which are currently incompletely understood and which cannot therefore be treated as formally modular.

Another approach is termed the “strip down” approach. This currently finds application in the production of useful secondary metabolites in Streptomyces spp. Here, genes and other genetic material not essential for growth are removed (“stripped out”), and those for selected biosynthetic pathways reintroduced individually. For example, Komatsu et al. (2010) PNAS 107 (6): 2646-2651 describe the construction of an engineered bacterium for heterologous expression of genes encoding secondary metabolite biosynthesis involving systematic deletion of nonessential genes from the genome of the industrial microorganism Streptomyces avermitilis.

The “strip down” approach requires methods for identifying genes, which are inessential (and so metabolically costly and therefore disadvantageous) for survival and/or growth under selected conditions.

Moreover, the approach would benefit from complementary functional genomic analyses to also identify genes which are advantageous for the proposed biotechnological applications. In the latter case, a “strip down/rev-up” approach could then be employed to minimize the metabolic burden arising from non-essential (disadvantageous) genes, while amplifying the beneficial effect of genes which directly or indirectly contribute to the biotechnological application (i.e. advantageous genes).

Transposon directed insertion-site sequencing (TraDIS—see Langridge et al. (2009) Genome Research 19: 2308-2316) has recently been described and used to identify: (a) essential genes; (b) genes advantageous (but not essential) for growth; (c) genes disadvantageous for growth under particular conditions; and (d) genes involved in conferring tolerance to certain conditions (“niche-specific” essential genes). Similar techniques have been described in e.g. Gawronski et al. (2009) PNAS 106: 16422-16427; Goodman et al. (2009) Cell Host Microbe 6: 279-289; van Opijnen et al. (2009) Nat. Methods 6: 767-772 and Gallagher et al. (2011) mBio 2(1):e00315-10, and such techniques are now collectively dubbed “Tn-seq” methods.

The present inventors have now discovered that TraDIS can be improved to provide an extremely elegant solution to the problem of unequivocally identifying both disadvantageous and advantageous genes for the purposes of bacterial bioengineering. This is achieved via the use of activating transposons (hereinafter abbreviated “Tn_{A}”). These transposons comprise a promoter such that transposon insertion into bacterial DNA at a suitable insertion site increases the transcription of a gene at or near that insertion site. Mutagenesis with Tn_{A} therefore yields insertionally-inactivated mutants (in which the Tn_{A} has disrupted gene expression, typically, but not necessarily, after insertion into the coding region on either strand) as well as insertionally-activated mutants (typically, but not necessarily, where the transposon has inserted upstream of a gene such that the promoter drives high level transcription (and consequently produces higher levels of expression) of the gene.

### SUMMARY OF THE INVENTION

According to the present invention, there is provided a process for producing a mutant bacterium which exhibits improved survival and/or growth under a selected growth condition, the process comprising the steps of:
(a) generating a pool of mutant bacteria by transposon mutagenesis with an activating transposon (Tn_{A}), wherein the Tn_{A} comprises an outward-facing promoter (Tn_{A}P) capable of increasing transcription of a gene at or near its insertion site in the DNA of said bacterium;
(b) growing bacteria from the mutant pool under the selected growth condition and under one or more reference conditions to produce two or more test cultures; and
(c) sequencing mRNA transcripts produced by Tn_{A}P in each of said test cultures to produce an mRNA transcript profile for each of the test cultures; and
(d) comparing the mRNA transcript profiles of the test cultures to identify a first class of genes which are disadvantageous for growth and/or survival under the selected growth condition and a second class of genes which are advantageous for growth and/or survival under the selected growth condition.

The mRNA transcript profile may comprise a determination of the sequence of one or more antisense transcripts arising from TnA insertion into an insertion site within a noncoding, anti-sense strand of the DNA of said bacterium. Such antisense transcripts may suppress gene expression, for example they may suppress expression of genes by binding to complementary mRNA encoded by the corresponding coding (sense) strand of the DNA of said bacterium.

Typically, Tn_{A} insertions associated with the first class of (disadvantageous) genes occur in the coding region (so that the gene is insertionally inactivated by the Tn_{A}) or outside of the coding region, but on the complimentary DNA strand to the coding sequence (causing either generation of antisense RNA or promoter activity disruption), whereas Tn_{A} insertions associated with the second (advantageous) class of genes occur upstream of the coding region in an orientation whereby the promoter of the Tn_{A} drives elevated transcription (and so expression) of the gene.

The process may further comprise the step of providing an engineered mutant bacterium in which at least one of said disadvantageous genes is removed, suppressed or disrupted and/or at least one of said advantageous gene is overexpressed, such that the mutant bacterium exhibits improved survival and/or growth under the selected growth condition. In such embodiments, a plurality of said disadvantageous genes may be removed or disrupted while a plurality of advantageous genes is overexpressed.

In this way, a mutant bacterium which is significantly better adapted to the selected growth condition (and so to the proposed biotechnological use) may be engineered or selected.

In such embodiments, the process may further comprise isolating and culturing the engineered mutant bacterium and then subjecting it to a further round of mutagenesis, culture and comparison (as defined in steps (a)-(d), above), and may optionally further comprise the step of providing a second round engineered mutant bacterium in which at least one of said further disadvantageous genes is removed or disrupted and/or at least one of said further advantageous gene is overexpressed, such that the mutant bacterium exhibits further improved survival and/or growth under the selected growth condition relative to the engineered mutant bacterium produced after the first round of mutagenesis.

Thus, the process of the invention is preferably iterative, and may comprise yet further rounds of mutagenesis and iterative application of steps (a) to (d) (as defined above) to provide a third, fourth, fifth (or greater) round mutant bacterium which exhibits yet further improved survival and/or growth in the presence of said environmental challenge relative to the engineered mutant bacterium of the preceding round.

In such embodiments, the selected growth condition applied during each round may be the same or different.

Other aspects and preferred embodiments of the invention are defined and described in the other claims set out below.

### BRIEF DESCRIPTION OF THE DRAWINGS

FIG. 1 shows a genetic map of plasmid pAMICS1-Cm-PrrnB.

FIG. 2 shows results from an analysis of transcription of genomic regions.

FIG. 3 shows results from an analysis of transcription.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term gene is a term describing a hereditary unit consisting of a sequence of DNA that occupies a specific location on a chromosome or plasmid and determines a particular characteristic, or group of characteristics, in an organism. A gene may determine a characteristic of an organism by specifying a polypeptide chain that forms a protein or part of a protein (structural gene); or encode an RNA molecule; or by specifying nucleic acid that forms a structural entity that influences, or in any way, regulates the operation of other genes or repress such operation (e.g. by acting in cis); or affect phenotype by some other as yet undefined mechanism.

The term genomic DNA is a term of art used herein to define chromosomal DNA as distinct from extrachromosomally-maintained (e.g. plasmid) DNA.

The term genome is a term of art used herein to define the entire genetic complement of an organism, and so includes chromosomal, plasmid, prophage and any other DNA or RNA acting a the genetic material.

The term Gram-positive bacterium is a term of art defining a particular class of bacteria that are grouped together on the basis of certain cell wall staining characteristics. Examples including Streptococcus spp., Staphylococcus spp., Listeria spp., Bacillus spp., Clostridium spp., Enterococcus spp. and Lactobacillus spp.)., actinomycetes (actinobacteria) including Actinomyces spp., Arthrobacter spp., Corynebacterium spp., Frankia spp., Micrococcus spp., Micromonospora spp., Mycobacterium spp., Nocardia spp., Propionibacterium spp. Bifidobacteriium spp and Streptomyces spp.

The term low G+C Gram-positive bacterium is a term of art defining a particular subclass class of evolutionarily related bacteria within the Gram-positives on the basis of the composition of the bases in the DNA.

The term high G+C Gram-positive bacterium is a term of art defining a particular subclass class of evolutionarily related bacteria within the Gram-positives on the basis of the composition of the bases in the DNA.

The term Gram-negative bacterium is a term of art defining a particular class of bacteria that are grouped together on the basis of certain cell wall staining characteristics.

Examples of Gram-negative bacterial of the Enterobacteriaceae including: Klebsiella, Escherichia, and Enterobacter as well as other Gram negative bacterial genera such as: Neisseria Acinetobacter, Pseudomonas and Burkholderia, and Bacteroides.

Selection of Growth Conditions

The processes of the invention permit the engineering of mutant bacteria which exhibit improved survival and/or growth under selected growth conditions, and involve detecting differences in the distribution and/or frequency of Tn_{A} insertions under a selected growth condition relative to one or more reference conditions.

The growth condition is selected according to the desired biotechnological application of the engineered bacteria ultimately produced: Any condition is suitable providing that differences between the selected and reference growth conditions drive a shift in the distribution of recoverable Tn_{A} insertion mutants in a test culture derived from the initial pool of Tn_{A} insertion mutants.

In certain embodiments, the selected growth condition is characterized by the presence of one or more selective agents which are absent (or present at a different, e.g. lower or higher, concentration in the reference condition(s)). In this context, the term “selective agent” is used in a broad sense to cover any agent (or combination of agents) which causes a change in the distribution and/or frequency of genomic Tn_{A} insertions when used in the process of the invention.

Such agents therefore include without limitation: environmental pollutants; toxins; antibiotics; carbon sources; nitrogen sources; energy sources; other microbes (e.g. yeasts, viruses, bacteria and/or plants); pH; pressure; temperature; salt concentration; pesticides; radioactive material; hydrocarbons; oil residues; industrial waste products; medical waste products; wastewater residues and the like.

Exemplary, non-limiting selective agents for use in providing the selected growth conditions of the processes of the invention according to the intended biotechnological use of the engineered bacteria are listed below:

Bioremediation—arsenic, metals, for example heavy metals and in particular lead, mercury uranium, palladium, chromium and cadmium; polynuclear aromatic hydrocarbons, chlorinated solvents, phenols, oils, pesticides and phosphates.

Microbial enhanced oil recovery—Crude oil and heavy oil fractions

Sewage treatment—nitrites, ammonia, phosphates and oestrogen-like compounds.

Food production—Lactose and acetic acid.

Biofuel production—Carbon dioxide, hydrogen, sunlight, oxygen, cellulose and hemicellulose.

Energy Generation—Waste water, marine sediment, freshwater sediment, river water, acetate, propionate and butyrate.

Bio-production and vaccines—Luria-Bertani or LB broth, Terrific Broth (TB), 2YT or chemically defined media.

Bio-digestion/biodegradation—Straw fractions, cellulosic waste and plastics.

Probiotics—Competition with other bacteria, for example Bifidobacterium spp and members of the Firmicutes phylum and/or any environmental condition relevant to the gut; e.g. nutrient utilization such as lactose, or pH.

Treatment of infection—Pathogenic bacteria, plants, the environment of the rhizosphere, Agrobacterium tumefaciens, animals, Staphylococcus aureus, human tissue and Clostridium difficile, human commensal bacteria or human mutualistic bacteria.

Bacteria for Use in the Methods of the Invention

The processes of the invention may be applied to the engineering of any bacterium. The bacterium selected will depend inter alia on the intended biotechnological use.

Thus, the processes of the invention find application in the identification of the production of antibiotics or antibiotics or their targets in: (a) Gram-positive, Gram-negative and/or Gram-variable bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, the methods of the invention are applied to bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorfen); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundii); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylori); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morganii); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. serotypes. Typhi and Typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. Y. pestis).

Exemplary, non-limiting bacteria for use in the processes of the invention according to the intended biotechnological use are listed below:

Bioremediation—Acinetobacter, Pseudomonas, Alcaligenes, Sphingomonas, Rhodococcus, Mycobacterium, Geobacter, Cupriavidus and Desulfovibrio.

Microbial enhanced oil recovery—Acinetobacter, Bacillus, Pseudomonas, Rhodococcus, Arthrobacter, Klebsiella and Clostridium.

Sewage treatment—Acinetobacter, Nitrobacter, Nitrococcus and Nitrospira.

Food production—Acetobacter.

Biofuel production—Ralstonia eutropha, Halanaerobium hydrogeniformans, Escherichia coli, Cyanobacteria, Clostridium acetobutylicum, Zymomonas mobilis and Caldicellulosiruptor obsidiansis.

Energy generation—Geobacter, Desulfuromonas, Proteobacterium, Pelobacter Thauera, Bacillus and Dechloromonas

Bio-production—Escherichia coli, Bacillus brevis, Bacillus megaterium, Bacillus subtilis, Caulobacter crescentus, Streptomyces,

Bio-digestion/biodegradation—Ralstonia eutropha, Halanaerobium hydrogeniformans, Escherichia coli, Cyanobacteria, Clostridium acetobutylicum, Zymomonas mobilis, Caldicellulosiruptor obsidiansis,

Vaccines—Caulobacter crescentus, Escherichia coli, Salmonella

Probiotics—Bifidobacterium, members of the Firmicutes phylum, human commensal bacteria and human mutualistic bacteria.

Treatment of Infection—competing “good” bacteria (e.g. human commensal bacteria or human mutualistic bacteria), plants, avirulent strains of Agrobacterium, Staphylococcus hominis and Bifidobacterium.

Genomic Engineering

The process of the invention may comprise the step of providing an engineered mutant bacterium in which at least one of said disadvantageous genes is removed or disrupted and/or at least one of said advantageous gene is overexpressed, such that the mutant bacterium exhibits improved survival and/or growth under the selected growth condition.

With regard to the removal or disruption of disadvantageous/inessential genes, various experimental procedures for chromosomal gene deletion/replacement in bacteria, which enable the specific substitution of targeted genome sequences with copies of those carrying defined mutations, are known in the art.

Two methods are of particular utility: the first (“in-out”) method is based on integration of vector DNA into the bacterial chromosome and subsequent resolution of the co-integrate. The second (“linear fragment” or recombineering) method is based on homologous recombination mediated by short homology arms at the ends of linear DNA molecule.

These methods are reviewed in e.g. Madyagol et al. (2011) Folia Microbiol 56: 253-263, the disclosure of which is hereby incorporated herein by reference.

Such methods may be used to delete large tracts of the bacterial genome, and if used to eliminate all (or substantially all) genes inessential for the desired biotechnological application (i.e. all dispensable genes that would otherwise impose a metabolic burden upon the bacterial cell), the process may be termed “genome minimization”.

In some embodiments, the mutant bacterium may be engineered to have a “minimized” genome that is smaller by 10%, 20%, 30%, 40%, 50%, 60%, 70%, 80%, 90% or more than 90% as compared to the wild type genome.

With regard to the overexpression, or suppression of advantageous or essential genes such that the mutant bacterium exhibits improved survival and/or growth under the selected growth condition, any of a wide variety of known techniques may be used, including inter alia the introduction of chemically-induced (or u.v. induced) point mutations, insertion of strong promoters on the coding or non-coding strand, ribosome binding sites, removal of repressor binding sites and optimization of codon usage.

Mutant Pools

The methods of the invention involve generating a pool of mutant bacteria by transposon mutagenesis. The size of the mutant pool affects the resolution of the method: as the pool size increases, more and more different genes with Tn_{A} insertions will be represented (and so effectively assayed). As the pool size decreases, the resolution of the method reduces, genes will be less effectively assayed, and more and more genes will not be assayed at all.

Ideally, the mutant pool generated in the methods of the invention is comprehensive, in the sense that insertions into every gene are represented. The number of Tn_{A} insertion mutants (i.e. the mutant pool size) required to achieve this depends on various factors, including: (a) the size of the bacterial genome; (b) the average size of the genes; and (c) any Tn_{A} insertion site bias.

With regard to the latter, some areas of bacterial genomes attract a low frequency of insertion (especially GC-rich regions). Thus, insertion frequencies and pool sizes large enough to ensure that insertions into insertion-refractory regions are preferred.

The insertion frequency will depend on the genome size and the regional GC skew but in general, a minimum insertion rate of one transposon per 25 bp is required to achieve a comprehensive pool/library, which typically entails a minimum pool size for bacteria having a genome size of 4 to 7 Mb of 0.5×10⁵ to 1×10⁵, for example 5×10⁵, preferably at least about 1×10⁶ mutants. In many cases, 1×10⁶ mutants will allow identification of 300,000 different insertion sites and correspond to 1 transposon insertion every 13 to 23 bp (or about 40-70 different insertion sites per gene).

However, the methods of the invention do not necessarily require a comprehensive mutant pool (in the sense defined above) in order to return useful information. Rather, pool sizes less than the ideal comprehensive pool may be used, provided that a reduction in resolution (and attendant failure to assay certain genes) can be tolerated. This may be the case, for example, where the method is designed to be run iteratively until the target is identified: in such embodiments the effective pool size grows with each iteration of the method.

Transposon Mutagenesis

Transposons, sometimes called transposable elements, are polynucleotides capable of inserting copies of themselves into other polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ends of RNA transcripts with 5′ ends often truncated.

Transposomes are transposase-transposon complexes wherein the transposon does not encode transposase activity. Thus, once inserted the transposon is stable. Preferably, in order to ensure mutant pool stability, the transposon does not encode transposase and is provided in the form of a transposome (i.e. as a complex with transposase enzyme), as described below.

As used herein, the term “activating transposon” (herein abbreviated “Tn_{A}”) defines a transposon which comprises a promoter such that transposon insertion increases the transcription of a gene at or near the insertion site. Examples of such transposons are described in Troeschel et al. (2010) Methods Mol Biol. 668:117-39 and Kim et al. (2008) Curr Microbiol. 57(4): 391-394.

The activating transposon/transposome can be introduced into the bacterial genome (including chromosomal and/or plasmid DNA) by any of a wide variety of standard procedures which are well-known to those skilled in the art. For example, Tn_{A} transposomes can be introduced by electroporation (or any other suitable transformation method).

Preferably, the transformation method generates 1×10³ to 5×10³ transformants/ng DNA, and such transformation efficiencies are generally achievable using electroporation. Alternatively, transposon mutagenesis using Tn_{A} may be performed in vitro and recombinant molecules transformed/transfected into bacterial cells. In such embodiments, transposomes can be prepared according to a standard protocol by mixing commercially available transposase enzyme with the transposon DNA fragment. The resulting transposomes are then mixed with extra-chromosomal DNA of interest to allow transposition, then the DNA is introduced into a host bacterial strain using electrotransformation to generate a pool of extra-chromosmal DNA transposon mutants.

In embodiments where mutagenesis is performed in vitro, it is possible to mix transposomes with genomic DNA in vitro and then introduce the mutagenized DNA (optionally, after fragmentation and/or circularization) into the host bacterial strain (e.g. by electroporation) whereupon endogenous recombination machinery incorporates it into the genome. Such an approach may be particularly useful in the case of bacteria which are naturally competent (e.g. Acinetobacter spp.) and/or can incorporate DNA via homologous crossover (e.g. double crossover) recombination events.

Activating Transposons for Use in the Methods of the Invention

Any suitable activating transposon may be used in the methods of the invention. Suitable transposons include those based on Tn3 and the Tn3-like (Class II) transposons including γδ (Tn1000), Tn501, Tn2501, Tn21, Tn917 and their relatives. Also Tn10, Tn5, TnphoA, Tn903, bacteriophage Mu and related transposable bacteriophages. A variety of suitable transposons are also available commercially, including for example the EZ-Tn5™<R6Kγori/KAN-2> transposon.

Preferred transposons are those which carry antibiotic resistance genes (which may be useful in identifying mutants which carry a transposon) including Tn5, Tn10 and TnphoA. For example, Tn10 carries a tetracycline resistance gene between its IS elements while Tn5 carries genes encoding polypeptides conferring resistance to kanamycin, streptomycin and bleomycin. Other suitable resistance genes include those including chloramphenicol acetyltransferase (conferring resistance to chloramphenicol).

It is of course possible to generate new transposons by inserting different combinations of antibiotic resistance genes between IS elements, or by inserting combinations of antibiotic resistance genes between transposon mosaic ends (preferred), or by altering the polynucleotide sequence of the transposon, for example by making a redundant base substitution or any other type of base substitution that does not affect the transposition or the antibiotic resistance characteristics of the transposon, in the coding region of an antibiotic resistance gene or elsewhere in the transposon. Such transposons are included within the scope of the invention.

In many embodiments, a single transposon is used to generate the mutant pool. However, as explained above, the number of Tn insertion mutants (i.e. the mutant pool size) required to achieve a comprehensive pool or library depends inter alia on any Tn insertion site bias. Thus, in cases where the transposon insertion site bias occurs, two or more different transposons may be used in order to reduce or eliminate insertion site bias. For example, a combination of two different transposons based on Tn5 and Tn10 may be employed.

Promoters for Use in Activating Transposons

The nature of the promoter present in the Tn_{A} is dependent on the nature of the transposon and the ultimate bacterial host. Generally, an efficient, outward-oriented promoter which drives high level transcription of DNA near or adjacent to the insertion site is chosen.

The promoter may include: (a) a Pribnow box (−10 element); (b) a −35 element and/or (c) an UP element.

For example, the lac promoter can be used with the EZ-Tn5™<R6Kγori/KAN-2> transposon, and such constructs are suitable for assay of e.g. Escherichia coli, Enterobacter spp. and other members of the family Enterobacteriaceae such as Klebsiella spp. Other suitable promoters include: rp/J (large ribosomal subunit protein; moderate strength promoter); tac (artificial lac/trp hybrid; strong promoter) and rrnB (ribosomal RNA gene promoter; very strong promoter).

Use of Multiple Tn_{A}/Multiple Promoters

In some embodiments of the invention, the bacterial genome is probed with a mixture of different activating transposons which have outward facing promoters of different strengths. In some circumstances, a broader range of genes involved in product production, resistance and/or survival and/or growth are recovered if a mixture of activating transposons with at least three different promoters of progressively decreasing strength are employed to generate the mutant pool.

In such circumstances, the use of a plurality of activating transposons with promoters of varying strength ensures that transposon insertions into substantially all genes involved in growth and/or survival are represented in the initial mutant pool, since transposon insertion can now result in gene activation to yield an appropriate level of transcription (neither too high, nor too low). Thus, the effect of the presence of antibiotic during subsequent culture of the mutant pool on transposon distribution can be studied (and the identity of the relevant gene target(s) thereby determined).

In such embodiments, a wide variety of promoters may be used provided that at least three different promoters are used wherein the relative strength of said promoters is: Tn_{A}P1>Tn_{A}P2>Tn_{A}P3; such that transposon insertion into bacterial DNA generates a pool of mutant bacteria in which one or more genes are transcribed from Tn_{A}P1, one or more genes are transcribed from Tn_{A}P2 and one or more genes are transcribed from Tn_{A}P3.

Preferably, Tn_{A}P1 is a strong promoter, Tn_{A}P2 a medium-strength promoter and Tn_{A}P3 a weak promoter in the mutagenized bacteria under the conditions used for growth of the mutant pool in the presence of the selective condition as defined in “selection of growth conditions”. In some embodiments, the relative transcription initiation rate of Tn_{A}P1 is at least 3 times, at least 100 times, at least 1000 times or at least 10000 times higher than that of Tn_{A}P3 under these conditions.

Each promoter includes: (a) a Pribnow box (−10 element); (b) a −35 element and (c) an UP element. Those skilled in the art are able to readily identify promoters having the required relative strengths by sequence analysis and/or in vitro or in vivo assays using expression constructs.

For example, suitable promoters can be engineered or selected as described in Rhodius et al. (2011) Nucleic Acids Research: 1-18.

Moreover, the rapid application of massively parallel or next generation sequencing to RNA-seq is now providing a wealth of high-resolution information of transcript start sites at a genomic level, which greatly simplifies the identification of promoter sequences in any given Gram-negative bacterium. This permits the construction of descriptive promoter models for entire genomes. RNA-seq also provides quantitative information on transcript abundance and hence promoter strength, which enables the construction of promoter strength models that can then be used for predictive promoter strength rankings (see Rhodium et al. (2011) Nucleic Acids Research: 1-18).

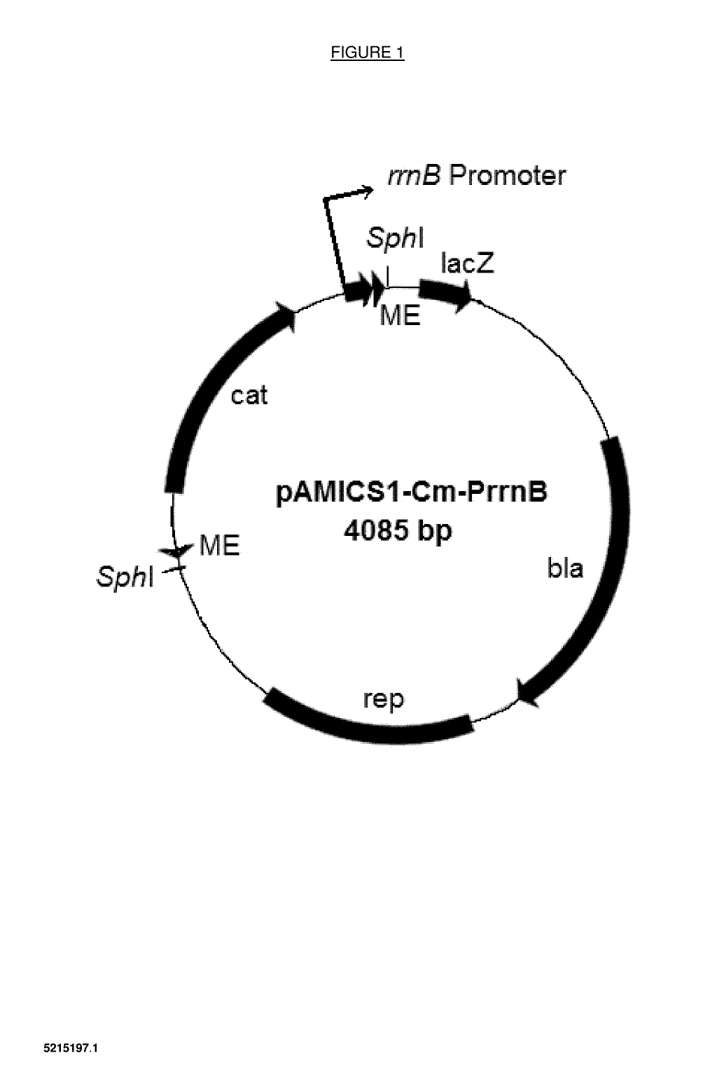

Suitable promoters can also be identified by assay. For example, a series of plasmids based on that shown in FIG. 1 can be used to test promoter strength empirically. Briefly, the promoter to be tested is placed upstream of an antibiotic resistance gene (in this case Kanamycin) and then transformed into the relevant bacteria. General cloning assembly and plasmid amplification can be carried out in E. coli (facilitated by the ampicillin resistance gene and the pBR322 ori) and the activity of the promoter in the target bacterium can then be assayed by generating a killing curve with Kanamycin—a very high level promoter gives more KmR expression and therefore survival at a higher antibiotic concentration. The plasmid series is designed to be modular so that the origin of replication, resistance gene(s) and promoter can be easily switched.

Suitable promoters include the E. coli rpIJ (large ribosomal subunit protein; moderate strength promoter); tac (artificial lac/trp hybrid; strong promoter) and rrnB (ribosomal RNA gene promoter; very strong promoter) promoters.

As used herein, the terms P_{rplJ} and P_{rrnB} specifically refer to the E. coli promoters for the 50S ribosomal subunit protein L10 and 16S ribosomal RNA genes, respectively. Orthologues of these (and other) E. coli promoters from other Gram-negative bacteria can also be used, including in particular the orthologous Pseudomonas aeruginosa or Acinetobacter baumannii promoters.

For example, the orthologous Acinetobacter baumannii gene corresponding to the E. coli rrnB P_{rrnB} has the gene symbol A1S_r12 and encodes the Acinetobacter baumannii 16S ribosomal RNA gene, so that the corresponding orthologous promoter is herein designated P_{(A1S}_{_}ᵣ₁₂₎. Thus, when the method is applied to Acinetobacter baumannii, Tnp, may be P_{(A1S}_{_}ᵣ₁₂₎.

Similarly, when the method of the invention is applied to Pseudomonas aeruginosa, Tn_{P2} may be the 16S ribosomal RNA gene promoter from P. aeruginosa (i.e. Ps.P_{rrnB}) while Tn_{P3} may be selected from the rpsJ (small (30S) ribosomal subunit S10 protein) gene promoter from P. aeruginosa (i.e. Ps.P_{rpsJ}) and the E. coli P_{rrnB}.

RNA Transcript Profiling

Sequencing mRNA transcripts produced by Tn_{A}P in each of said test cultures permits an mRNA profile to be produced for each of the test cultures.

The transcript profile may comprise:
the sequences of said mRNA transcripts produced by TnAP; and/or
The start and stop sequence location of said mRNA transcript
the lengths of said mRNA transcripts produced by TnAP; and/or
the relative abundance of said mRNA transcripts produced by TnAP; and/or
the site of transcription on the bacterial DNA; and/or
whether the mRNA transcripts produced by TnAP is sense or antisense with respect to the bacterial DNA; and/or
whether the mRNA transcripts produced by TnAP correspond to ORFs with respect to the bacterial DNA; and/or
whether the mRNA transcripts produced by TnAP encode bacterial proteins and/or protein domains.

In preferred embodiments, said mRNA transcript profile comprises a determination of the sequence of one or more antisense transcripts arising from TnA insertion into an insertion site within a noncoding, anti-sense strand of the DNA of said bacterium.

Any suitable high-throughput massively parallel sequencing technique can be used, including those based on sequencing-by-synthesis (SBS) and nanopores, and there are many commercially available sequencing platforms that are suitable for use in the methods of the invention. SBS-based sequencing platforms are particularly suitable for use in the methods of the invention: for example, the Illumina™ system is generates millions of relatively short sequence reads (54, 75 or 100 bp) and is particularly preferred.

Other suitable techniques are based on reversible dye-terminators. Here, DNA molecules are first attached to primers on a slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labeled nucleotides then the dye along with the terminal 3′ blocker is chemically removed from the DNA, allowing a next cycle.

Other systems capable of short sequence reads include SOLiD™ and Ion Torrent technologies (both sold by Applied Biosystems™). SOLiD™ technology employs sequencing by ligation. Here, a pool of all possible oligonucleotides of a fixed length are labelled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion Torrent Systems Inc. have developed a system based on using standard sequencing chemistry, but with a novel, semiconductor based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

Functional Assessment of Putative Essential Genes

The profiling of the mRNA transcripts may comprise a determination of whether the mRNA transcripts produced by Tn_{A}P encode bacterial proteins and/or protein domains.

This may comprise characterization of the mRNA transcripts by various techniques which directly or indirectly assess the function of the RNA per se (or the encoded proteins).

Suitable techniques include bioinformatics, where the (full or partial) sequence of the putative essential gene is used to interrogate sequence databases containing information from the bacterium assayed and/or other species in order to identify genes (e.g. orthologous genes in other species) for which essential biochemical function(s) have already been assigned and/or which have been shown to be essential.

Suitable bioinformatics programs are well known to those skilled in the art and include the Basic Local Alignment Search Tool (BLAST) program (Altschul et al. (1990) J. Mol. Biol. 215: 403-410 and Altschul et al. (1997) Nucl. Acids Res. 25: 3389-3402). Suitable databases include, for example, EMBL, GENBANK, TIGR, EBI, SWISS-PROT and trEMBL.

Alternatively, or in addition, the (full or partial) sequence of the gene is used to interrogate a sequence database containing information as to the identity of essential genes which has been previously constructed using the conventional Tn-seq methods described in the prior art (e.g. as described in Gawronski et al. (2009) PNAS 106: 16422-16427; Goodman et al. (2009) Cell Host Microbe 6: 279-289; van Opijnen et al. (2009) Nat. Methods 6: 767-772; Langridge et al. (2009) Genome Research 19: 2308-2316; Gallagher et al. (2011) mBio 2(1):e00315-10) and/or the techniques described in WO 01/07651 (the contents of which are hereby incorporated by reference).

Despite the presence of a promoter within the inserted sequence, many Tn_{A} insertions will disrupt gene/DNA function and allow identification of essential/important DNA regions, as in standard Tn-seq (including TraDIS). However, some transposons will be positioned appropriately with respect to specific DNA regions, whereby transcription of those specific regions, driven by the inserted promoter, is enhanced significantly compared to endogenous transcription. By growing the mutant pool under different conditions and repeating the sequencing it is possible to observe changes in the number of reads, indicating not only which DNA region contributes to growth and/or survival, but also the relative contribution. The higher levels of specific antibiotic target transcription (driven by the transposon-inserted promoters) will favour bacterial survival and link insertion site to DNA region by proximity.

The position of the inserted promoter can be assessed with respect to its contribution to increased transcription of relevant downstream DNA sequences. A mathematically/technically straightforward bioinformatics component of this technique permits recognition of the contribution of the inserted promoter sequence to transcription of the putative gene. For example, the partial gene transcript may still encode enough information to allow translation of a truncated, but functional protein. Bioinformatics would allow the effects of transcriptional read through on genes downstream of the gene adjacent to the inserted transposon to be considered, where there is there no defined RNA transcription termination sequence.

For example, a transposon/promoter upstream of genes A, B and C may generate a polycistronic transcript of all three genes (A-C), upstream of B a polycistronic transcript of genes B and C and upstream of C just gene C. If the reads for the first two transposons were high and the third low in antibiotic then the antibiotic target would be gene B.

Biotechnological Applications of the Engineered Mutant Bacteria of the Invention

These include: bioremediation; microbial enhanced oil recovery; wastewater treatment; sewage treatment; food production; energy production; bio-production; bio-digestion/biodegradation; vaccines; biosensors; probiotics; biocatalysis; biological control; crop production and drug delivery vehicles.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1: Production of Mutant Bacteria which Exhibit Improved Survival and/or Growth in the Presence of Fosfomycin

(i) Construction of Activating Transposon (Tn_{A})

Plasmids were constructed which incorporate amplifiable nucleotide sequences which act as transposons. The elements of the transposon include the 19 bp mosaic ends which are recognised by a specific transposase enzyme and delimit the transposon, an antibiotic-resistance gene to select for transformants that have resulted from transposition, and an outward oriented promoter at one end of the transposon to activate expression of target genes adjacent to the transposon insertion site.

Alternative plasmids have been constructed with different outward oriented promoters from different genes from E. coli, Acinetobacter, or Pseudomonas. Table 1 provides details of the different promoters used. In addition, different host species bacteria require different antibiotic resistance genes to select for transformants, e.g. chloramphenicol resistance may be used in E. coli, kanamycin resistance in Acinetobacter spp. and gentamicin resistance in Pseudomonas spp. Details of different resistance genes used are given in Table 2.

FIG. 1 is a genetic map of plasmid pAMICS1-Cm-PrmB. The transposon is flanked by the mosaic ends, ME, and the outward oriented rrnB promoter in indicated. Other components of the plasmid outside of the transposon are from the plasmid vector pBluescript (Agilent), lacZ, beta-galactosidase subunit; bla, beta-lactamase coding for ampicillin resistance; rep, pBR322 replication origin; cat, chloramphenicol acetyltransferase coding for chloramphenicol resistance.

Other suitable plasmid-derivatives are similar to pAMICS1-Cm-PrrnB, except that the outward-oriented rrnB promoter is substituted for promoters listed in Table 1 and the chloramphenicol resistance determinant is substituted for those listed in Table 2.

TABLE 1

| Promoter | Bacteria | Source | Accession No. |
|---|---|---|---|
| tac | E. coli | pKK223 | X95387 |
| rplJ | E. coli | E. coli UTI89 | CP000243 |
| rrnB | E. coli | E. coli UTI89 | CP000243 |
| rrn | Acinetobacter baumannii | ATCC 17978 | CP000521 |
| rplJ | Acinetobacter baumannii | ATCC 17978 | CP000521 |
| rrnB | Pseudomonas aeruginosa | ATCC 15692 | AE004091 |
| rpsJ | Pseudomonas aeruginosa | ATCC 15692 | AE004091 |
| rplK | Pseudomonas aeruginosa | ATCC 15692 | AE004091 |

TABLE 2

| | Source | Source of | |
|---|---|---|---|
| Resistance gene | plasmid | sequence | Used in: |
| Kanamycin | Kanamycin | | Acinetobacter baumannii |
| | GenBlock | | |
| Chloramphenicol | pACYC184 | X06403.1 | E. coli |
| Gentamicin | pFastBac1 | Invitrogen | Pseudomonas aeruginosa |
| | | | and Rhodopseudomonas |
| | | | palustris |

Preparation of Transposomes

Transposon DNA fragments for use in transposon mutagenesis were PCR amplified using specific oligos. For generation of the PCR templates, pAMICS1-Cm-P plasmids were digested with the restriction endonuclease SphI (obtained from New England Biolabs) which cleaves the plasmid at either side of the mosaic ends and the 1.2 kb transposon fragments generated were isolated following electrophoresis through 1% agarose using a “MinElute gel extraction kit” (obtained from QIAGEN). This step prevents the later generation of transformants due to carry-through of plasmid template in the transposon mutagenesis process, ensuring the transformants obtained are transposon mutants and not due to transformants harbouring the plasmid used as the template to generate the transposon fragments.

PCR amplification of the transposon fragments was carried out using “PfuUltra II Fusion” DNA polymerase, which is a “proof-reading” DNA polymerising enzyme (obtained from Agilent). The PCR amplified, de-salted DNA fragment was then treated with polynucleotide kinase to phosphorylate the 5′-DNA ends. Transposomes are then prepared using the purified transposon fragment and recombinant Tn5 transposase.

Preparation of Electrocompetent Escherichia coli

Electrocompetent cells for transposon mutagenesis were prepared by inoculating 5 ml of LB-broth with Escherichia coli (ST131) colonies streaked from a frozen stock. This culture was incubated overnight with shaking to promote growth. Typically 2 ml was then used to inoculate 400 ml of pre-warmed 2×YT broth and the culture incubated at 3TC until the optical density measured at a wavelength of 600 nm was between 0.2 and 0.3. Cells were harvested by centrifugation and washed by resuspending in 10% glycerol. Cells were retrieved by centrifugation and again resuspended in 10% glycerol. This step was repeated until the cells had been washed at least 3 times. Finally, cells were resuspended using a volume of 10% glycerol which is 1/1000 of the volume of the 2×YT culture (e.g. 400 μl).

Generation of Escherichia coli Transposon Mutant Pools

60 μl of the cell suspension described in the previous step were mixed with 0.2 μl of transposomes, and the mixture electroporated using a “Gene II Pulser” (BioRad). Typically, electroporation cuvettes of 2 mm electrode gap were used with electroporation settings of 2.5 kV, 25 ρF and 200Ω. These settings resulted in a time constant of 4.8 to 5 msec. 1 ml of S.O.C. broth was then added to the cuvette and, after mixing, the cell suspension transferred to a fresh tube and incubated for 1 h 30 mins at 37° C. The cell suspension was then spread on LB-agar plates supplemented with the appropriate concentration of chloramphenicol (e.g. 7.5 μl/ml), and the agar plates were incubated at 37° C. to allow growth of chloramphenicol-resistant transformant colonies resulting from transposition of the transposon into the bacterial genome.

The numbers of colonies obtained were then estimated so as to provide an approximate number of transposon mutants. Colonies were then harvested from the agar plates by resuspending in a volume of LB-broth using a bacteriological spreader. To this cell suspension glycerol was added to a concentration of 15% and the suspension split into aliquots for storage at −80° C.

Determination of mRNA Transcript Profile from Escherichia coli Transposon Mutant Pools

This step assays every gene in the bacterial genome for contribution to growth under any chosen condition. It allows the ordering of every gene according to relative contribution to the growth condition, starting with genes that contribute significantly, through genes that provide no significant contribution, through to the genes that are disadvantageous for growth of the bacterial cell under the chosen conditions.

In this example, the selected growth condition was the presence of the antibiotic Fosfomycin.

Pools of the transposon mutant bacteria are grown in the presence of the antibiotic. Typically, this may be performed in 10 ml broth cultures to which 10⁸ individual bacterial transposon mutants have been added from an aliquot of the −80° C. frozen stock. Several cultures should be included each with a different concentration of antibiotic. For example, concentrations which are ½× the minimum inhibitory concentration (MIC), 1×MIC and 2×MIC may be performed. Experiments with transposon mutant pools harbouring transposons with the differing promoters (tac, rpIJ or rrnB) may be performed in a single pool, or more cultures may be included if the different mutant pools are to be investigated separately.

Following incubation of the cultures at 3TC overnight in media luria broth or broth supplemented with 60 μM or 150 μM Fosfomycin, 0.1 ml of culture was then transferred to a fresh 10 ml LB-broth supplemented with the same concentration of Fosfomycin under investigation, and this culture incubated for at least 6 h to allow growth of the bacteria.

Bacteria were then harvested from the culture by centrifugation and total RNA extracted using RNeasy mini-kit (Qiagen) RNA samples were 3′ tagged by ligation, of a 5′ phosphorylated, 3′ dideoxy terminated DNA oligonucleotide RP-1 (phosphorylation-5′-AAC CTG GAC ATA CGC ATC CG(ddA)-3′) to the 3′ end of the RNA using T4 RNA ligase (NEB). The 3′ tagged RNA molecules were then converted to cDNA, using a reverse complement of the RP-1 oligo, RP-1-rev (5′-CGG ATG CGT ATG TCC AGG TT-3′) by incubating with M-MLV reverse transcriptase RNaseH minus (Promega) at 30° C. for 30′, 45° C. for 30′ and 50° C. for 30′, then treated with 100 U/μl RNaseA and 0.02 U/μl RNaseH to degrade any surviving RNA. The cDNA was then amplified by PCR with phusion DNA polymerase (Thermo) using an oligo with sequence specificity to the transposon in a region common to all promoters, TN-5-fwd (5′-CGA CTC ACT ATA GGG AGA TGT-3′) and RP-1-rev as the reverse primer. PCR reactions were cleaned up using a Wizard SV reaction purification kit (Promega) and then sequencing performed on an Ion Torrent (Life Technologies) Proton sequencer, after fragmented library preparation. The sequencing runs were used to determine the position and abundance of the transposon inserts within the genome and visualised using Artemis genome browser (Sanger).

Identification of Genes Advantageous for Growth in the Presence of Fosfomycin

Mapping of sequence reads to an E. coli reference genome may be used to indicate the major loci to which mapped very large numbers of reads, indicating the survival of significant numbers of mutants with transposon insertions at these loci and therefore that these loci are important for the survival of mutants in the presence of Fosfomycin.

### Example 2: Production of Mutant Bacteria which Exhibit Improved Survival and/or Growth at Low pH

The ability of bacteria to grow in acid conditions is of direct use for the biotechnology industry. The E. coli transposon library as described in Example 1 is also used for this example as the mutant pools

Mutant pools of bacteria are grown in laboratory media at increasingly low pH. The pH of the media is manipulated by altering the ratio of hydrogen phosphates as the buffering system. Typically, this is performed in 10 ml broth cultures to which 10⁸ individual bacterial transposon mutants are added. In the experiment several cultures are grown in media with a different buffered pH. For example, cultures at pH 6, 5, 4 and 3.5 (normal pH range for E. coli K12 is pH 4.5 (J. Bacteriol. March 1994 vol. 176 no. 6 1729-1737) may be performed. Experiments with transposon mutant pools harbouring transposons with the differing promoters (tac, rpIJ or rrnB) may be performed in a single pool, or more cultures may be included if the different mutant pools are to be investigated separately.

Following incubation of the cultures, at 3TC overnight in buffered YT, acid tolerant mutants are identified by sequencing. Bacteria are harvested from the culture by centrifugation and mRNA extracted and sequenced. Stable genetically engineered mutants may be prepared and used to generate a second E. coli transposon mutant pool. The second library, in the acid tolerant E. coli background, is exposed to progressively lower pH—this may be carried out as iterative rounds of selection until required acid tolerance is achieved.

### Example 3: Identification of Essential Genes/Genes Becoming Essential on Ceftriaxone Exposure

Libraries of transposon mutants in bacteria were generated as described above to produce at least 3 libraries with different strength outward facing promoters. These libraries were then pooled to ensure equal spread of the mutations and grown in the presence of ceftriaxone at 2× the MIC at 37° C. in appropriate growth media at a ratio that ensured 100's of copies of each transposon for 16 h.

The cells were then subcultured by 1:100 dilution into fresh media containing 2×MIC ceftriaxone and then grown into exponential phase (OD₆₀₀ of 0.4-0.5, usually taking 3-4 hours) at 37° C. At this point 2×0.5 ml of culture was harvested by centrifugation and both total RNA and genomic DNA extraction using RNeasy mini-kit (Qiagen) or Wizard SV genomic DNA kit (Promega).

DNA was quantified by UV spectrophotometry and 4.5 μg fragmented by sonication on a Covaris M220 and library prepared by end repair and ligation of specific amplification tags to the fragmented DNA, followed by PCR amplification for transposon sequences and the ligation tags. The libraries were then sequenced on Ion torrent proton sequencers. RNA samples were 3′ tagged with either a polyA tail using E. coli poly(A) polymerase (NEB) or by ligation, of a 5′ phosphorylated, 3′ dideoxy terminated DNA oligonucleotide RP-1 (phosphorylation-5′-AAC CTG GAC ATA CGC ATC CG(ddA)-3′), to the 3′ end of the RNAusing T4 RNA ligase (NEB). All oligonucleotide used were ordered from Integrated DNA technologies with HPLC purification. The 3′ tagged RNA molecules were then converted to cDNA, using a poly dT₂₀ oligo for E. coli poly(A) polymerase tagged RNA, or alternatively reverse complement of the RP-1 oligo, RP-1-rev (5′-CGG ATG CGT ATG TCC AGG TT-3′) by incubating with M-MLV reverse transcriptase RNaseH minus (Promega) at 30° C. 30′, 45° C. for 30′ and 50° C. for 30′, then treated with 100 U/μl RNaseA and 0.02 U/μl RNaseH to degrade any surviving RNA. The cDNA was then amplified by PCR with phusion DNA polymerase (Thermo) using an oligo with sequence specificity to the transposon in a region common to all promoters, TN-5-fwd (5′-CGA CTC ACT ATA GGG AGA TGT-3′) and either RP-1-rev or poly dT₁₂ as the reverse primer. PCR reactions were cleaned up using a Wizard SV reaction purification kit (Promega) and then library preparation in the same manner as the genomic DNA. The sequencing runs were used to determine the position and abundance of the transposon inserts within the genome and visualised using Artemis genome browser (Sanger).

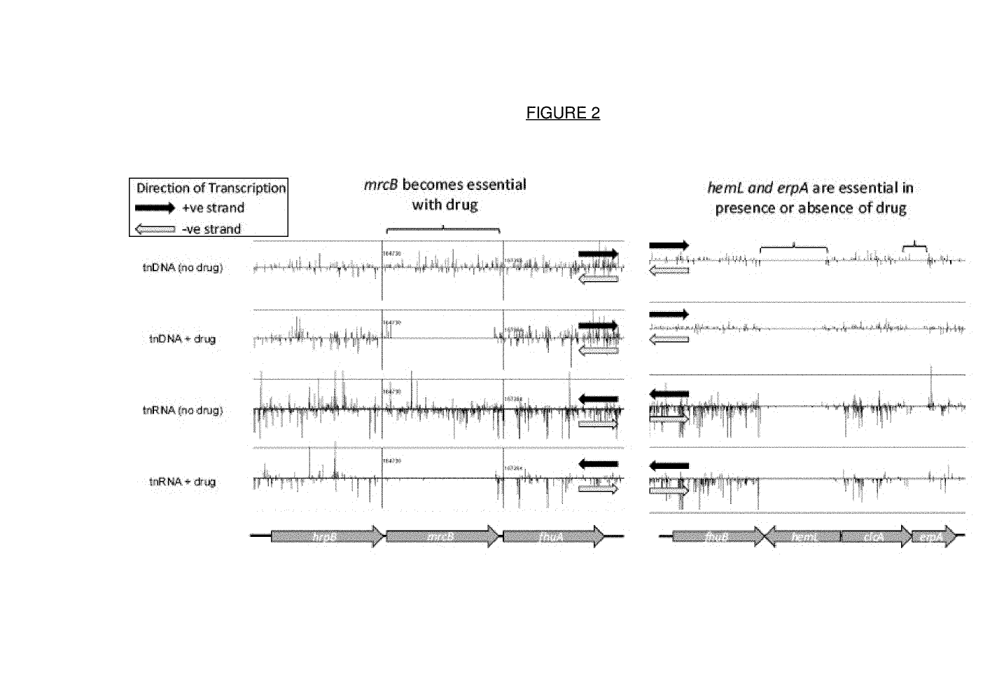

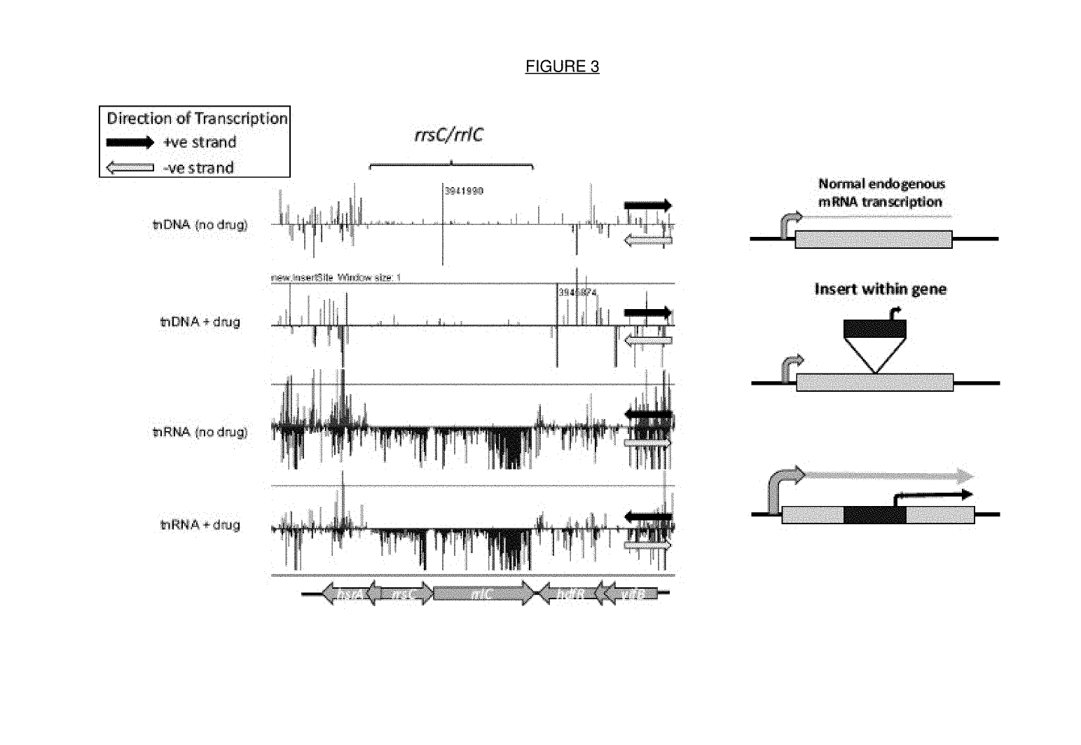

As shown in FIG. 2, the RNA sequencing provides similar positional data to the DNA sequencing for the location of transposons and in many cases few inserts are seen within genes essential for growth or that become essential with drug treatment. Additionally it also shows levels of transcription of genomic regions. For example were genomic inserts are rare there are still abundant RNA inserts detected suggesting very high transcription in this region, either the gene is always heavily transcribed, or the response to the insertion is massive increases in expression to try and compensate for the reduced/loss of functionality of the disrupted gene, as well as some transcription from the transposon itself (FIG. 3).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A process for producing a mutant bacterium which exhibits improved survival and/or growth under a selected growth condition, the process comprising the steps of:
(a) generating a pool of mutant bacteria by transposon mutagenesis with an activating transposon (Tn_{A}), wherein the Tn_{A} comprises an outward-facing promoter (Tn_{A}P) capable of increasing transcription of a gene at or near its insertion site in the DNA of said bacterium;
(b) growing bacteria from the mutant pool under the selected growth condition and under one or more reference conditions to produce two or more test cultures; and
(c) sequencing mRNA transcripts produced by Tn_{A}P in each of said test cultures to produce an mRNA transcript profile for each of the test cultures; and
(d) comparing the mRNA transcript profiles of the test cultures to identify a first class of genes which are disadvantageous for growth and/or survival under the selected growth condition and a second class of genes which are advantageous for growth and/or survival under the selected growth condition.

2. The process of claim 1, further comprising the step of providing an engineered mutant bacterium in which at least one of said disadvantageous genes is removed or disrupted and/or at least one of said advantageous gene is overexpressed, such that the mutant bacterium exhibits improved survival and/or growth under the selected growth condition.

3. The process of claim 2 wherein a plurality of said disadvantageous genes is removed or disrupted.

4. The process of claim 2 wherein a plurality of said advantageous genes is overexpressed.

5. The process of claim 2 further comprising culturing the engineered mutant bacterium and then applying steps (a)-(d) to said engineered mutant bacterium to identify further first class of genes which are disadvantageous for growth and/or survival under the selected growth condition and a further second class of genes which are advantageous for growth and/or survival under the selected growth condition.

6. The process of claim 5 further comprising the step of providing a second round engineered mutant bacterium in which at least one of said further disadvantageous genes is removed or disrupted and/or at least one of said further advantageous gene is overexpressed, such that the mutant bacterium exhibits improved survival and/or growth under the selected growth condition relative to the engineered mutant bacterium.

7. The process of claim 6 comprising one or more further rounds of mutagenesis and iterative application of steps (a) to (d) to provide a third or greater round mutant bacterium which exhibits improved survival and/or growth in the presence of said environmental challenge relative to the engineered mutant bacterium of the previous round.

8. The process of claim 2 wherein the removal and/or disruption of said disadvantageous genes comprises genome minimization.

9. The process of claim 8 wherein said genome minimization comprises integration of plasmid DNA into the bacterial chromosome and subsequent resolution of the cointegrate, or homologous recombination mediated by short homology arms at the ends of a linear DNA.

10. (canceled)

11. The process of claim 1 further comprising the step of introducing at least one heterologous gene into the bacterium.

12. The process of claim 11 wherein said heterologous gene is advantageous for growth and/or survival under the selected growth condition.

13. The process of claim 11 further comprising the step of introducing a heterologous gene cluster into the bacterium

14. The process of claim 13 wherein the heterologous gene cluster encodes a biosynthetic pathway or a biodegradative pathway.

15. The process of claim 14 wherein the biosynthetic pathway yields a secondary metabolite.

16. (canceled)

17. The process of claim 11 wherein said heterologous gene encodes a therapeutic protein.

18. The process of claim 17 wherein said therapeutic protein is:
(a) an enzyme;
(b) an antibody;
(c) an antigen;
(d) a toxin;
(e) a ligand-binding protein;
(f) an antibiotic;
(g) a peptide; or
(h) a cytokine.

19. The process of claim 1 wherein the selected growth condition comprises the presence of:
(a) an environmental contaminant;
(b) an industrial waste product;
(c) a medical waste product;
(d) a drug or candidate drug;
(e) a selected carbon source; or
(f) one or more other organisms.

20. The process of claim 19 wherein said one or more other organisms are:
(a) human pathogens;
(b) animal pathogens; or
(c) plant pathogens.

21. The process claim 1 wherein the pool of mutant bacteria comprises at least 0.5×10⁵ mutants.

22-43. (canceled)

44. A mutant bacterium obtainable, or obtained by, a process as defined in claim 1.
